(19) United States
(12) Patent Application Publication
(10) Pub. No.: US 2015/0051463 A1
(43) Pub. Date: Feb. 19, 2015

(54) Oximetry Signal, Pulse-Pressure Correlator

(71) Applicant: Guy P. Curtis, San Diego, CA (US)

(72) Inventor: Guy P. Curtis, San Diego, CA (US)

(21) Appl. No.: 14/334,335

(22) Filed: Jul. 17, 2014

Related U.S. Application Data
(60) Provisional application No. 61/867,005, filed on Aug. 16, 2013.

(51) Int. Cl. A61B 5/00; A61B 5/0285; A61B 5/021

(52) U.S. Cl. CPC A61B 5/7246; A61B 5/02116; A61B 5/0285; A61B 5/746
USPC 600/324

(57) ABSTRACT

A system and method are provided for using an oximeter to take blood pressure readings for an extended period of time. Calibration of the oximeter for this purpose requires use of a sphygmomanometer to determine a sequence of blood pressure readings taken for a patient over a sphygmomanometer duty cycle. During the duty cycle, readings for both blood pressure (sphygmomanometer) and blood flow amplitude (oximeter) are taken simultaneously at predetermined time intervals (e.g. patient pulse rate). These readings then determine an operational ratio between the two that can be used to translate pulse magnitude readings of the oximeter for presentation as blood pressure readings. Operationally, variations from the patient's systolic pressure can then be continuously monitored in real time.

This application claims the benefit of U.S. Provisional Patent Application Ser. No. 61/867,005, filed Aug. 16, 2013. The entire contents of Application Ser. No. 61/867,005 are hereby incorporated by reference herein.

### FIELD OF THE INVENTION

The present invention pertains to systems and methods for continuously monitoring the blood pressure of a patient over an extended period of time. More particularly, the present invention pertains to systems and methods wherein a patient's blood flow, as measured by an oximeter, is evaluated in terms of blood pressure readings. The present invention is particularly, but not exclusively, useful for systems and methods wherein the incremental changes in blood pressure, that are measured by a sphygmomanometer during a duty cycle of the sphygmomanometer, are correlated with changes in pulse amplitude as measured by an oximeter during the same duty cycle, for subsequent use of the oximeter in measuring a patient's blood pressure.

### BACKGROUND OF THE INVENTION

An ability to continuously monitor the blood pressure of a patient over an extended period of time is clinically beneficial for several reasons. At present, the most commonly accepted methodology for measuring a patient's blood pressure involves the use of a sphygmomanometer. In its use, a sphygmomanometer will provide blood pressure pulse measurements during its duty cycle that include a systolic measurement and a diastolic measurement. In detail, the systolic measurement provides a blood pressure reading for the phase of the patient's heartbeat when the heart muscle contracts and pumps blood from the chambers into the arteries. On the other hand, the diastolic measurement provides a blood pressure reading for the phase of the heartbeat when the heart muscle relaxes and allows the chambers of the heart to fill with blood. Typically, these measurements are referenced together and evaluated as systolic/diastolic. Although a sphygmomanometer is both accurate and reliable, its use can be cumbersome. Consequently, the repetitive use of a sphygmomanometer to obtain continuous readings over an extended period of time may be problematic.

Apart from the sphygmomanometer, an oximeter is a well-known and commonly used device for measuring blood pulse amplitudes. Specifically, an oximeter is typically used to monitor a patient's pulse rate. To do this, a sensor is merely clamped onto the finger of a patient and the oximeter is thereafter capable of continuously monitoring blood flow pulse amplitudes. This can be done for an extended period of time, without interruption.

With the above in mind, several general considerations are helpful for an appreciation of the present invention. These considerations, which are all patient specific, include:
A patient's diastolic pressure will remain substantially constant during a stabilized condition. On the other hand, the systolic pressure will vary most significantly.
Physiologically, absent an anomaly, the impedance to blood flow in a patient's cardiovascular system will generally remain substantially constant over an extended period of time.
Pulse amplitude signals taken by an oximeter are directly proportional to blood flow level.

In light of the above, it is an object of the present invention to provide a system and a method for continuously monitoring blood flow in the vasculature of a patient. Another object of the present invention is to provide a system and method for using blood pressure pulse measurements, taken by a sphygmomanometer, to calibrate an oximeter for subsequent use in monitoring the blood pressure of a patient. Still another object of the present invention is to provide a system and method for simultaneously monitoring blood pressure and blood flow pulse amplitudes over an extended period of time which is easy to use, simple to implement and comparatively cost effective.

### SUMMARY OF THE INVENTION

In accordance with the present invention, a system and method are provided to continuously monitor blood flow in a patient for an extended period of time. In particular, this monitoring is accomplished using a conventional oximeter as the sensor, and using a sphygmomanometer to periodically calibrate the oximeter. As envisioned for the present invention, after the oximeter has been calibrated it can be employed to continuously generate blood flow pulse amplitude signals that are indicative of blood pressures generated by the patient's heart beat.

For purposes of the present invention, a calibration of the oximeter begins by first connecting both the oximeter and the sphygmomanometer to the patient. In this combination, the sphygmomanometer is used for measuring a blood pressure pulse magnitude pₛ for each pulse of the patient's heart. Simultaneously, the oximeter is used for measuring a blood flow pulse amplitude pₒ. Both measurements are taken contemporaneously during a sphygmomanometer duty cycle which extends between a systolic pressure p_{s(systolic)} and a diastolic pressure p_{s(diastolic)} of the patient.

During the sphygmomanometer duty cycle that is used for calibrating the oximeter, the respective magnitude and amplitude measurements for pₛ₀ (sphygmomanometer) and pₒ (oximeter) are received as input at a computer. After completion of the duty cycle, these measurements are used by the computer to establish an operational ratio, pₒ/pₛ, that is based on contemporary measurements of pₛ and pₒ during the duty cycle. In detail, the operational ratio, pₒ/pₛ, is preferably established as follows. For an n number of pulses during the sphygmomanometer duty cycle, successively different blood pressure magnitude measurements pₛₙ are taken by the sphygmomanometer for each pulse (heart beat). Simultaneously, corresponding blood flow amplitude measurements pₒₙ are also taken by the oximeter. An average change in blood pressure pulse magnitude Δpₛ [Δpₛ=(Σ Δpₛₙ)n] is then calculated, and it is compared with an average change in pulse amplitude Δpₒ [Δpₒ=(Σ Δpₒₙ)n]. The computer then uses the ratio Δpₒ/Δpₛ to establish the operational ratio pₒ/pₛ. As will be appreciated by the skilled artisan, conventional curve fitting techniques can be employed in this process. In any event, as implied above, the operational ratio pₒ/pₛ is then used to determine a blood pressure value pₛ that is based on pulse amplitudes pₒ that are measured in real time.

In an operation of the present invention, a monitor, which is connected to the computer, is used to continuously compare pulse amplitude signals pₒ from the oximeter with the base amplitude p_{o(base)}. Specifically, this comparison is done in real time, to detect variations of pₒ from the base amplitude p_{o(base)} as an indicator of changes in blood flow and, hence, changes in blood pressure. Further, an alarm can be initiated by the computer to indicate whenever a pulse amplitude signal pₒ has a maximum/minimum value that differs from the base amplitude p_{o(base)} by a predetermined value. For instance, these predetermined values can be based on the operational ratio pₒ/pₛ to cause an alarm with a positive change (maximum value) of more than 60 mmHg or a negative change (minimum value) of more than 40 mmHg in blood pressure pₛ.

Other aspects of the present invention that are noteworthy include the notion that during a calibration of the oximeter, blood pressure pulse magnitudes pₛ and blood flow pulse amplitudes pₒ are taken during the sphygmomanometer duty cycle at a same selected point in each pulse of the patient's heart. Also, the operational ratio Δpₒ/Δpₛ that results from these measurements is always patient specific. Furthermore, the operational ratio Δpₒ/Δpₛ for calibrating the oximeter is preferably recalculated at least every hour.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a schematic depiction of an employment of a system in accordance with the present invention;

FIG. 2 is a calibration graph showing sphygmomanometer measurements (blood pressure pulse magnitude) and corresponding oximeter measurements (blood flow pulse amplitude) taken at a same time during a sphygmomanometer duty cycle;

FIG. 3 is a graph showing a relationship between blood pulse amplitude and blood pressure for use by a computer when correlating pulse amplitude signals measured by an oximeter as blood pressure readings; and

FIG. 4 is a depiction of a linear scale for use by the computer when comparing the pulse amplitude signals with a reference value, in real time, to monitor blood flow.

### DESCRIPTION OF THE PREFERRED EMBODIMENTS

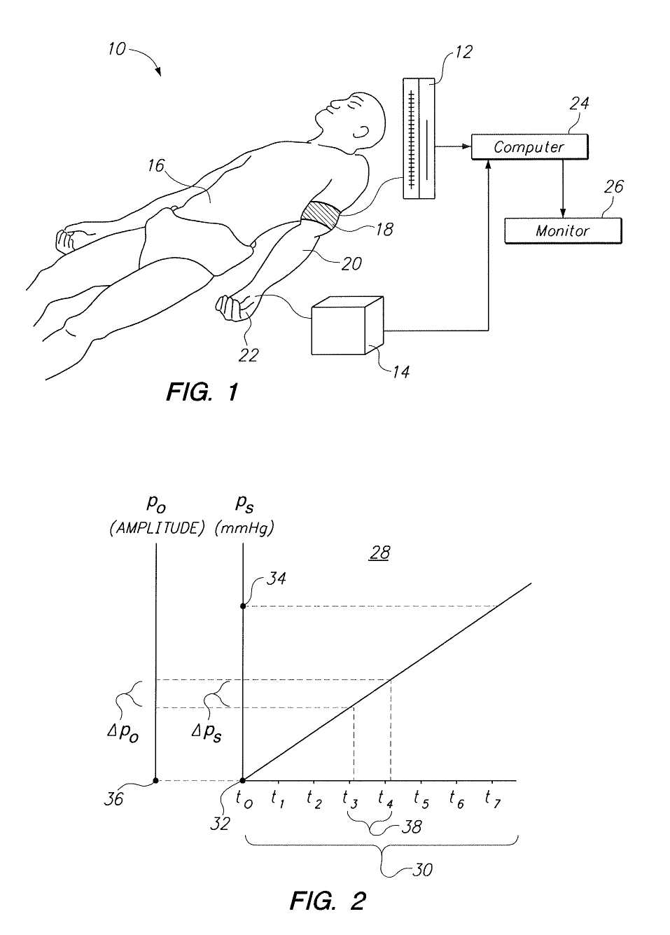

Referring initially to FIG. 1, a system for continuously monitoring blood flow in the vasculature of a patient is shown, and is generally designated 10. As shown, the system 10 includes both a sphygmomanometer 12 and an oximeter 14. In FIG. 1, these components of the system 10 are shown in use together, and are connected with a patient 16 for the purpose of taking simultaneous measurements. In this combination, the sphygmomanometer 12 is used for the purpose of taking blood pressure pulse measurements, pₛ. Thus, it will typically include a pressure cuff 18 which is placed on an arm 20 of the patient 16. On the other hand, the oximeter 14 is used for the purpose of taking blood flow pulse amplitude measurements, pₒ. Thus, it will typically include a clamp (not shown in detail) that is connected directly with a finger 22 of the patient 16.

As is well known, the sphygmomanometer 12 and the oximeter 14 are normally employed independently, for different purposes. The present invention, however, envisions their concurrent use during a set-up (i.e. calibration) of the system 10. In particular, the set-up of system 10 is undertaken to calibrate blood flow pulse amplitudes measured by the oximeter 14, with blood pressure measurements from the sphygmomanometer 12. The specific purpose here is to calibrate the oximeter 14 for a subsequent, independent use of the oximeter 14, by itself, for monitoring the blood pressure of patient 16, without the sphygmomanometer 12.

FIG. 1 also shows that both the sphygmomanometer 12 and the oximeter 14 are connected with a computer 24. A monitor 26 is also connected with the computer 24. Further, it is to be appreciated that the monitor 26 will include a visual display (not shown) which provides continuous, real-time information from the oximeter 14 and from the computer 24 regarding the blood pressure of the patient 16. An important aspect of the present invention is that this information can be provided over an extended period of time.

FIG. 2 shows a calibration graph 28 which illustrates an exemplary correspondence between blood pressure pulse magnitudes pₛ and simultaneous blood flow pulse amplitudes pₒ. For a set-up of the system 10, measurements of both pₛ and pₒ are respectively taken by the sphygmomanometer 12 and the oximeter 14 during a same sphygmomanometer duty cycle 30.

As indicated by the graph 28, exemplary blood pressure measurements (i.e. pₛ) are sequentially taken for each heart beat during the duty cycle 30 (e.g. at times t₀ through t₇). Importantly, during the duty cycle 30, the particular blood pressure measurement which is taken at time tₒ, at point 32 on graph 28, corresponds with the systolic pressure, p_{s(systolic)}, of the patient 16. Similarly, the measurement at point 34 on graph 28 which is taken at time t₇, corresponds to the diastolic pressure, p_{s(diastolic)}, of the patient 16. Further, for reasons more clearly established below, the systolic pressure, p_{s(systolic)}, of the patient 16 (point 32) is correlated with a simultaneous measurement taken by the oximeter 14, which is represented by the point 36 in graph 28. The blood flow pulse amplitude measurement which is indicated at point 36, is then subsequently used as a base amplitude measurement, p_{o(base)}.

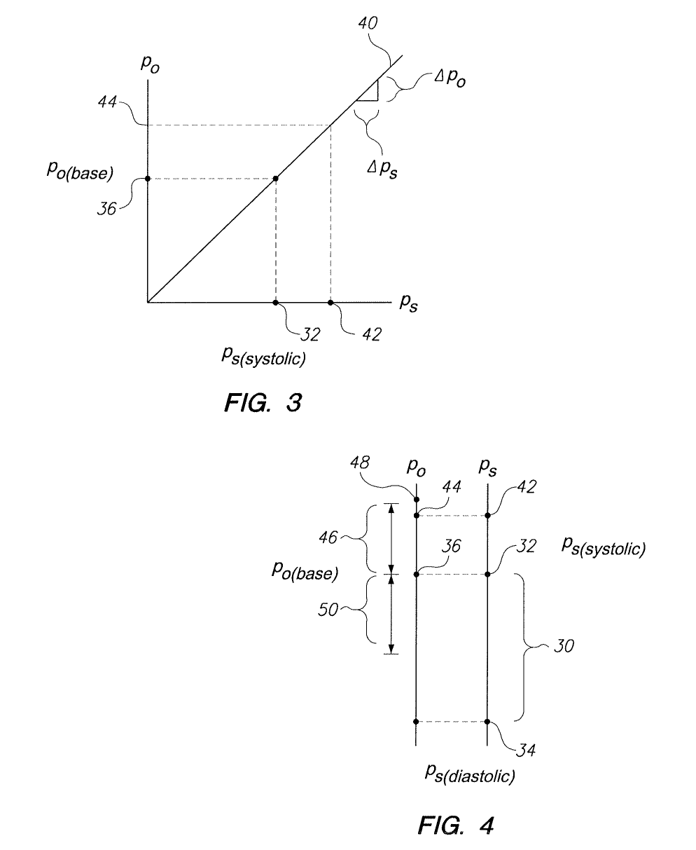

A correlation between blood pressure pulse magnitudes, pₛ, and blood flow pulse amplitudes, pₒ, is based on changes Δpₛ and Δpₒ between the respective measurements taken at successive time tₙ and tₙ₊₁ during the duty cycle 30. For instance, referring to FIG. 2 it will be seen that at the time t₃ in the duty cycle 30, a reading pₛ₃ is obtained for a blood pressure measurement, and a reading pₒ₃ is obtained for a blood flow pulse amplitude measurement. Subsequently, at time t₄, measurements pₛ₄ and pₒ₄ are respectively taken. Thus, during the time interval 38 between t₃ and t₄, shown in FIG. 2, a change in blood pressure pₛ₄−pₛ₃=Δpₛ₃ and a change in pulse amplitude pₒ₄−pₒ₃=Δpₒ₃ are determined. A series of an n number of such measurements taken over a duty cycle 30 can then be represented by the line graph 40 in FIG. 3 using well known curve fitting techniques.

In detail, the line graph 40 is based on a comparison between an average change in blood pressure pulse magnitude Δpₛ [Δpₛ=(Σ Δpₛₙ)n] and an average change in blood flow pulse amplitude Δpₒ [Δpₒ=(Σ Δpₒₙ)n]. For example, with n=8, the averages will be based on measurements taken sequentially at times t₀ through t₇ over the sphygmomanometer duty cycle 30. The result here is the ability to mathematically determine an operational ratio Δpₒ/Δpₛ (e.g. the slope of the line graph 40) that is patient specific, and that can be used for determining a blood pressure value pₛ based on changes in pulse amplitude pₒ.

In overview, each blood pressure pulse magnitude pₛ and each blood flow pulse amplitude pₒ is taken at a selected point in each heart pulse of the patient 16 (e.g. at a time tₙ). These measurements are taken during the sphygmomanometer duty cycle 30, and are provided as input to the computer 24 for calculating the operational ratio Δpₒ/Δpₛ.

For an operation of the system 10, the oximeter 14 is calibrated, and periodically recalibrated as necessary, to correlate pₒ with pₛ. Specifically, this is done in accordance with a methodology for determining the operational ratio Δpₒ/Δpₛ as disclosed above. Using a calibrated oximeter 14, the monitor 26 is then continuously available for checking the blood flow/pressure condition of the patient 16. As will be appreciated with reference to FIG. 4, the system 10 will monitor for when a change in blood pressure causes the pulse amplitude pₒ measured by the oximeter 14 to vary from the base amplitude p_{o(base)} by a predetermined value.

By way of example, while cross referencing FIG. 3 with FIG. 4, consider a change in pₛ from point 32 to point 42. For the system 10, this change in pₛ to the point 42 is indicated by a change in pₒ to the point 44 from the point 36 (i.e. p_{o(base)}) As shown in FIG. 4, this change keeps pₒ within a range 46 of predetermined value (e.g. where pₛ remains less than p_{s(systolic)}+60 mmHg). Otherwise, as intended for the present invention, when pₒ exceeds the value at point 48, pₛ will be greater than p_{s(systolic)}+60 mmHg and the system 10 can be set to alarm. On the other hand, also by way of example, when pₒ goes below p_{o(base)} and beyond a range 50 of predetermined value (e.g. where pₛ is below p_{s(systolic)}−40 mmHg), the system 10 can be set to alarm. As will be appreciated by the skilled artisan, the values given in this example can be varied as desired by the user. In any event, it is also to be appreciated that the operational ratio Δpₒ/Δpₛ will, preferably, be recalculated to recalibrate the oximeter 14 at least every hour.

While the particular Oximetry Signal, Pulse-Pressure Correlator as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A system for continuously monitoring blood flow in the vasculature of a patient which comprises:
a sphygmomanometer for measuring a blood pressure pulse magnitude pₛ for each pulse of the patient's heart during a sphygmomanometer duty cycle, wherein the sphygmomanometer duty cycle extends between a systolic pressure p_{s(systolic)} and a diastolic pressure p_{s(diastolic)};
an oximeter for measuring a blood flow pulse amplitude pₒ for each pulse of the patient's heart during the sphygmomanometer duty cycle, wherein pₛ and pₒ are measured simultaneously for each pulse;
a computer for establishing an operational ratio pₒ/pₛ based on contemporary measurements of pₛ and pₒ, and for identifying a base amplitude signal p_{o(base)} to correspond with the systolic pressure p_{s(systolic)} of the patient; and
a monitor connected to the computer for continuously comparing pulse amplitude signals pₒ from the oximeter with the base amplitude p_{o(base)}, in real time, to detect variations therebetween as an indicator of changes in blood pressure and blood flow.

2. A system as recited in claim 1 further comprising an alarm initiated by the computer for indicating when a pulse amplitude pₒ, measured by the oximeter, varies from the base amplitude by a predetermined value.

3. A system as recited in claim 2 wherein the predetermined value is based on the operational ratio pₒ/pₛ with a positive change of more than 60 mmHg and a negative change of more than 40 mmHg in blood pressure pₛ.

4. A system as recited in claim 1 wherein the oximeter is connected to the patient at a selected pulse pressure location of the patient.

5. A system as recited in claim 1 wherein, for an n number of pulses during a sphygmomanometer duty cycle, successively different blood pressure measurements pₛₙ are taken by the sphygmomanometer and corresponding blood flow measurements pₒₙ are taken by the oximeter to establish the operational ratio pₒ/pₛ.

6. A system as recited in claim 5 wherein, over the duty cycle, an average change in blood pressure pulse magnitude Δpₛ [Δpₛ=(Σ Δpₛₙ)n] is compared with an average change in pulse amplitude Δpₒ[Δpₒ=(Σ Δpₒₙ)n] over the duty cycle to determine an operational ratio Δpₒ/Δpₛ for determining a blood pressure value pₛ based on changes in pulse amplitude pₒ.

7. A system as recited in claim 6 wherein each blood pressure pulse magnitude pₛ and each blood flow pulse amplitude pₒ is taken at a selected point in each pulse of the patient's heart during the sphygmomanometer duty cycle.

8. A system as recited in claim 6 wherein each operational ratio Δpₒ/Δpₛ is patient specific.

9. A system as recited in claim 6 wherein the operational ratio Δpₒ/Δpₛ is recalculated to recalibrate the oximeter at least every hour.

10. A method for continuously monitoring blood flow in the vasculature of a patient which comprises the steps of:
measuring a blood pressure pulse magnitude pₛ, with a sphygmomanometer, for each pulse of the patient's heart during a sphygmomanometer duty cycle, wherein the sphygmomanometer duty cycle extends between a systolic pressure p_{s(systolic)} and a diastolic pressure p_{s(diastolic)};
measuring a blood flow pulse amplitude pₒ, with an oximeter, for each pulse of the patient's heart during the sphygmomanometer duty cycle, wherein pₛ and pₒ are measured simultaneously for each pulse;
establishing an operational ratio pₒ/pₛ based on contemporary measurements of pₛ and pₒ;
identifying a base amplitude signal p_{o(base)} to correspond with the systolic pressure p_{s(systolic)} of the patient; and
continuously comparing pulse amplitude signals pₒ from the oximeter with the base amplitude p_{o(base)}, in real time, to detect variations therebetween as an indicator of changes in blood pressure and blood flow.

11. A method as recited in claim 10 further comprising the step of initiating an alarm when a pulse amplitude pₒ, measured by the oximeter, varies from the base amplitude by a predetermined value.

12. A method as recited in claim 11 wherein the predetermined value is based on the operational ratio pₒ/pₛ with a positive change of more than 60 mmHg and a negative change of more than 40 mmHg in blood pressure pₛ.

13. A method as recited in claim 10 wherein, for an n number of pulses during a sphygmomanometer duty cycle, successively different blood pressure measurements pₛ, are taken by the sphygmomanometer and corresponding blood flow measurements pₒₙ are taken by the oximeter to establish the operational ratio pₒ/pₛ.

14. A method as recited in claim 13 wherein, over the duty cycle, an average change in blood pressure pulse magnitude Δpₛ [Δpₛ=(Σ Δpₛₙ)n] is compared with an average change in pulse amplitude Δpₒ [Δpₒ=(Σ Δpₒₙ)n] over the duty cycle to determine an operational ratio Δpₒ/Δpₛ for determining a blood pressure value pₛ based on changes in pulse amplitude pₒ.

15. A method as recited in claim 14 wherein each blood pressure pulse magnitude pₛ and each blood flow pulse amplitude pₒ is taken at a selected point in each pulse of the patient's heart during the sphygmomanometer duty cycle.

16. A method as recited in claim 10 further comprising the step of recalculating the operational ratio Δpₒ/Δpₛ to recalibrate the oximeter at least every hour.

17. A method as recited in claim 10 wherein the establishing step and the identifying step are accomplished by a computer.

18. A method as recited in claim 10 wherein the comparing step is accomplished by a computer with input from a monitor.

19. A non-transitory, computer-readable medium having executable instructions stored thereon that direct a computer system to perform a process that comprises: measuring a blood pressure pulse magnitude pₛ, with a sphygmomanometer, for each pulse of the patient's heart during a sphygmomanometer duty cycle, wherein the sphygmomanometer duty cycle extends between a systolic pressure p_{s(systolic)} and a diastolic pressure p_{s(diastolic)}, measuring a blood flow pulse amplitude pₒ, with an oximeter, for each pulse of the patient's heart, wherein pₛ and pₒ are measured simultaneously for each pulse; establishing an operational ratio pₒ/pₛ based on contemporary measurements of pₛ and pₒ; identifying a base amplitude signal p_{o(base)} to correspond with the systolic pressure p_{s(systolic)} of the patient; and continuously comparing pulse amplitude signals pₒ from the oximeter with the base amplitude p_{o(base)}, in real time, to detect variations therebetween as an indicator of changes in blood pressure and blood flow.

20. A medium as recited in claim 19 wherein the process further comprises: taking successively different blood pressure measurements pₛₙ and corresponding blood flow measurements pₒₙ, for an n number of pulses during a sphygmomanometer duty cycle, to establish the operational ratio pₒ/pₛ; and comparing an average change in blood pressure pulse magnitude Δpₛ [Δpₛ=(Σ Δpₛₙ)/n] with an average change in pulse amplitude Δpₒ [Δpₒ=(Σ Δpₒₙ)/n] over the duty cycle to determine the operational ratio Δpₒ/Δpₛ for determining a blood pressure value pₛ based on changes in pulse amplitude pₒ.
